(19) United States
(12) Patent Application Publication
(10) Pub. No.: US 2022/0133674 A1
(43) Pub. Date: May 5, 2022

(54) ORALLY INGESTED COMPOSITION FOR IMPROVING SLEEP

(71) Applicant: WELLNAS.CO., LTD, Tokyo (JP)

(72) Inventors: Masahiro KOYAMA, Tokyo (JP); Kozo NAKAMURA, Nagano (JP)

(21) Appl. No.: 17/433,900

(22) PCT Filed: Feb. 27, 2020
(86) PCT No.: PCT/JP2020/007950

§ 371 Date: Aug. 25, 2021

(30) Foreign Application Priority Data
Feb. 28, 2019 (JP) 2019-035956

(51) Int. Cl. A61K 31/221; A61K 9/00; A61K 36/81; A61K 36/899; A61P 25/20

(52) U.S. Cl. CPC A61K 31/221; A61K 9/0053; A61P 25/20; A61K 36/899; A61K 36/81

(57) ABSTRACT

The present invention addresses the problem of providing a novel orally ingested composition that contains a substance which is not conventionally known as an active component of a composition for improving sleep, and particularly, that can be easily taken orally and can improve sleep safely and easily. The present invention pertains to an orally ingested composition, particularly a food composition, for improving sleep and containing a choline ester as an active component In the present invention, the choline ester may be derived from an edible plant such as the fruit of the species Solanum melongena of the genus Solanum of the family Solanaceae and/or the green shoot of the tribe Bambuseae of the subfamily Bambusoideae of the family Poaceae.

### TECHNICAL FIELD

The present invention relates to an composition for oral ingestion for improving sleep comprising as an effective ingredient a choline ester, a compound in which choline is bound to an organic acid by ester bond.

### BACKGROUND ARTS

Sleep is a physiological phenomenon of great importance in animals. Fifty percent or more people of Japanese population suffer from insomnia. It is estimated that there is an economic loss of 6 trillion yen scale a year that includes decreased productivity due to insufficient sleep and health expenditure including hypnotics.

Sleep aid compositions using various food materials have been investigated so far. For instance, sleep aid compositions using theanine contained in tea leaves (Patent Reference 1), sesamin contained in sesame (Patent Reference 2), and lactoferrin obtained from mammalian milk, etc. (Patent Reference 3) have been proposed.

Acetylcholine, which is known to be an essential substance as a neurotransmitter for biological activity in mammals, has been implicated in sleep. However, this implication goes no further than an indication of a role of acetylcholine as an intracerebral active substance, as in the report that an injection of an acetylcholine receptor agonist to brain stem induces REM (rapid eye movement) sleep-like state (Non-Patent Reference 1). There has been no specific investigation about the relationship between acetylcholine and sleep when acetylcholine is orally ingested.

On the other hand, certain food plants such as fermented buckwheat (lactic acid fermentation product of Fagopyrum vulgare plant), aubergines and bamboo shoots contain choline esters such as acetylcholine at high concentrations. Orally ingested compositions for hypotensive or anti-stress purpose utilizing these have been proposed (Patent Reference 4 to 6).

### PRIOR ART REFERENCES

### Patent References

[Patent Reference 1] WO 01/74352

[Patent Reference 2] JP A 2010-285427

[Patent Reference 3] JP A 201843964

[Patent Reference 4] WO 2015/147251

[Patent Reference 5] JP A 2015-189745

[Patent Reference 6] WO 2018/070545

### NON-PATENT REFERENCES

[Non-Patent Reference 1] Niwa, Y. et al., “Muscarinic acetylcholine receptors Chrm1 and Chrm3 are essential for REM sleep”, Cell Reports, 24: 2731-2247 (2018).

### SUMMARY OF THE INVENTION

### Problems to be Solved by the Invention

The present inventors have aimed at solving distress of people who desire to improve sleep, improving productivity, and reducing economic loss such as health expenditures spent on hypnotics, etc., by providing to a market a composition that comprises a substance that has conventionally not been known as an effective ingredient for sleep aid composition.

Accordingly, an object of the present invention is to provide a composition that comprises a substance that has conventionally not been known as an effective ingredient for sleep aid composition, particularly a novel composition for oral ingestion that is easily capable of being orally ingested and that is capable of improving sleep in a safe and simple manner.

### Means to Solve the Problems

The present inventors made an intensive research in order to solve the above-described problem and in due course discovered that a sleep improving effect can be obtained by using a composition for oral ingestion comprising a choline ester. The present inventors further proceeded the research, and as a result brought the present invention to completion.

Accordingly, the present invention relates to the followings:
[1] A composition for oral ingestion for improving sleep comprising a choline ester as an effective ingredient
[2] The composition for oral ingestion according to [1] above, wherein the composition is a food composition.
[3] The composition for oral ingestion according to [1] or [2] above, wherein the choline ester is derived from a food plant
[4] The composition for oral ingestion according to [3] above, wherein the food plant is a fruit of the family Solanaceae, the genus Solanum, the species Solanum melongena, and/or a young bud of the family Poaceae, the subfamily Bambusoicleae, the tribe Bambuseae.
[5] The composition for oral ingestion according to any one of [1] to [3] above, wherein the choline ester comprises one or more selected from the group consisting of acetylcholine, butylcholine and propionylcholine.
[6] The composition for oral ingestion according to [5] above, wherein the choline ester does not comprise lactoylcholine.

[7] The composition for oral ingestion according to any one of [1] to [6] above, wherein daily intake of the choline ester is between 7.5 μg and 750 mg.

[8] The composition for oral ingestion according to any one of [1] to [7] above, wherein the improving sleep is an increase in sleeping time.

### Effects of the Invention

The present invention can provide a composition for oral ingestion that is capable of safely and simply improving sleep by using a choline ester as an effective ingredient In particular, by using a choline ester of food plant-origin, a sleep aid composition can relatively easily and extremely inexpensively be provided.

Moreover, because choline esters have been well-experienced as being eaten in food, the composition of the present invention is highly safe and can be utilized as a food composition and as a pharmaceutical composition for improving sleep.

### Mode for Carrying out the Invention

The present invention relates to a composition for oral ingestion for improving sleep comprising a choline ester as an effective ingredient.

The composition of the present invention can be used for increasing sleep time or for improving sleep cycle, etc., thereby being capable of improving sleep.

The composition of the present invention may be a food composition or a pharmaceutical composition. The composition of the present invention is a pharmaceutical composition, it may be a pharmaceutical composition for improving sleep or treating sleep disorder.

Choline ester, which is an effective ingredient of the composition of the present invention, that can be used may be derived from an animal, or derived from a plant, or derived from a microorganism, though it is preferred to use one derived from an organism that has been eaten by human. In particular, it is preferred to be derived from a food plant

The food plant is not particularly limited as long as it contain a choline ester. For instance, it includes food plants such as the family Cucurbitaceae (e.g., cucumber), the family Solanaceae (e.g., aubergine), the family Liliaceae (e.g., asparagus), the family Dioscoreaceae (e.g., Japanese yam), the family Brassicaceae (e.g., cabbage), the family Asteraceae (e.g., lettuce), the family Apiaceae (e.g., carrot), the family Rosaceae (e.g., apple), the family Vitaceae (e.g., grape), the family Leguminosae (e.g., alfalfa), the family Polygonaceae (e.g., buckwheat), and the family Poaceae (e.g., bamboo shoot). In view of acetylcholine content, the family Solanaceae the genus Solanum, the species Solanum melongena, and the family Poaceae, the subfamily Bambusoidecte, the tribe Bambuseae are preferred, and a fruit of the family Solanaceae, the genus Solanum, the species Solanum melongena and/or a young bud of the family Poaceae, the subfamily Bambusoideae, the tribe Bambuseae is preferred.

Among the breeds of the family Solanaceae, the genus Solanum, the species Solanum melongena, SENSHU MIZUNASU®, BATTEN NASU, KORYO SALADE NASU (also known as BINAN), HIGO-MURASAKI, OHNAGA NASU, CHIKUYO, etc. are preferred. SENSHU MIZUNASU®, BATLE,N NASU, KORYO SALADE NASU and HIGO-MURASAKI are preferred because they are capable of being eaten raw. HIGO-MURASAKI is particularly preferred.

The choline ester content in 100 g (raw weight) of the food material that can be used in the present invention is approximately as shown in Table 1.

TABLE 1

| Food material | Choline ester content in 100 g (raw weight) | |
|---|---|---|
| Aubergine | 6.12 | mg |
| Bamboo shoot | 14.3 | mg |
| Bell pepper | 5.95 × 10⁻³ | mg |
| Buckwheat sprout | 4.19 × 10⁻³ | mg |
| Grape | 3.15 × 10⁻³ | mg |
| Broccoli sprout | 3.11 × 10⁻³ | mg |
| Japanese yam | 2.87 × 10⁻³ | mg |

The choline ester that can be contained in the composition of the present invention includes acetylcholine, butylcholine, propionylcholine and lactoylcholine. The composition may comprise one or more among these. In particular, when the choline ester is derived from a plant, the composition of the present invention comprises one or more selected from the group consisting of acetylcholine, butylcholine and propionylcholine, but does not comprise lactoylcholine.

The composition of the present invention is adjusted such that daily intake of the choline ester is within a predetermined range.

In order to improve sleep, daily intake of the choline ester is adjusted within a range between 7.5 μg and 1,000 mg, preferably between 7.5 μg and 750 mg, particularly preferably between 15 μg and 750 mg. For instance, the choline ester content is adjusted within a range between 2.5 μg and 300 mg, preferably between 2.5 μg and 250 mg, particularly preferably between 5 μg and 250 mg in one package (e.g., in one capsule), which can be orally ingested once a day or divided into several times a day. Moreover, the choline ester may be provided in several packages such that its total amount is adjusted within the above-described range. In this case, the concentration of the choline ester is between 15 and 3,750 μg/g.

The composition of the present invention is preferably taken just before to 2 hours before turning in, particularly preferably from 30 minutes to 1 hour before turning in. For instance, it is preferred to be taken once a day before taming in for 5 to 30 days, more preferably for 5 to 14 days.

The composition of the present invention is adjusted to contain a predetermined choline ester content. In the composition of the present invention, a cut and divided fresh agricultural product, a frozen product, a freeze-dried product or an extract, etc., may be used. The composition of the present invention preferably is a composition consisting of freeze-dried powder and/or extract of a food plant.

The composition of the present invention may be provided in a form cut and divided for daily use such that it contains a daily intake of the choline ester, or individually packaged such as in a vacuum pack in order to prevent quality degradation or to prevent browning of fruit pulp.

Moreover, the composition of the present invention is preferably frozen. By freezing process, the activity of cholinesterase contaminating in the composition can be suppressed, retaining the choline ester for a prolonged period. The composition of the present invention is preferably a part of or whole frozen fruit of the family Solanareae, the genus Solanum, the species Solanum melongena.

The composition of the present invention can, in one embodiment, be produced by a method in which a food plant is heat-sterilized before being extracted to give juice which is then freeze-dried. It is also possible to add an excipient to the extracted juice and freeze-dry it. Various excipient such as dextrin, lactose, and microcrystalline cellulose can be used. The amount of the excipient to be added is between 5 and 75 wt %, preferably between 10 and 50 wt %, particularly preferably between 10 and 25 wt % to the weight of the extracted juice.

Moreover, the composition of the present invention can, in one embodiment, be produced by a method comprising preparing a freeze-dried and/or hot-air dried powder or an extract, and dispensing the freeze-dried and/or hot-air dried powder or the extract such that the choline ester content is a predetermined amount

Here, the predetermined amount may be between 7.5 μg and 1,000 mg, preferably between 7.5 μg and 750 mg, further preferably between 15 μg and 750 mg.

A method of producing a composition of the present invention may further comprise healing the food plant Healing may be carried out by heating it in a microwave oven or boiling it in hot water. For instance, when 100 g of the food plant is heated in a microwave oven at 550 W, it is heated for 1 to 15 minutes, preferably for 2 to 10 minutes, further preferably for 4 to 6 minutes. When being boiled in hot water, it is preferred to be heated in hot water at 90 to 100° C. By thus heating the food plant, it can be sterilized, and at the same time, the choline ester in the food plant can be increased.

Normally, the decomposition by microorganisms is avoided by freeze-drying or hot-air drying. The method of producing the composition of the present invention is, in one embodiment, characterized in that the method further comprises heat-processing the food plant (sterilizing and choline ester-increasing effects).

The method of producing the composition of the present invention may further comprise suspending the freeze-dried powder and/or hot-air dried powder of the food plant in water, and adding an acid to the obtained suspension. The pH of the acid-added suspension is adjusted at, for example, between 5.5 and 4.5, preferably between 5.4 and 4.6. Thus adjusting pH stabilizes choline ester and can provide a composition (suspension) suitable for long-term preservation.

The method of producing the composition of the present invention may comprise extracting the freeze-dried powder and/or hot-air dried powder of the food plant with ethanol or hydrous ethanol.

More specifically, the method of the present invention may comprise a choline ester-concentrated extract, which is obtained by adding ethanol or hydrous ethanol to the freeze-dried powder and/or hot-air dried powder of the food plant or by adding ethanol to fresh food plant, grinding it and removing the residue.

When extraction is performed with hydrous ethanol, ethanol concentration of the hydrous ethanol is not particularly limited, and it can appropriately selected in consideration of extracting rate and concentrating rate of the choline ester, etc. Ethanol concentration of the hydrous ethanol can be, for example, 10% (w/w) or higher , preferably from 10 to 99% (w/w), more preferably from 25 to 60% (w/w) or 95% (w/w) or higher, particularly preferably from 30 to 60% (w/w) or 99% (w/w) or higher.

L-ascorbic acid may be added o ethanol or hydrous ethanol used for extraction. For instance, it is added at between 1 and 5 wt %, preferably at 3 wt %.

The method of the present invention may comprise adjusting choline ester content of the extract to between 5 μg and 50 mg.

It is preferred that the dry powder pertaining to the composition of the present invention is powder which has passed through an appropriate mesh sieve. The composition of the present invention preferably consists of freeze-dried powder and/or hot-air dried powder that can pass through, for example, a 20-mesh sieve.

Here, the hot-air dried powder can be prepared, for example, by exposing the food plant to a hot blast at about 90° C. for 1 hour to 2 hours to dry it, grinding and powdering it

The composition of the present invention can be used as an effective ingredient in various functional health food or medicament composition.

In a case of food, it can be combined with an appropriate food additive and used as a composition for food. Moreover, not only as such a composition for food, it can also be provided in a form that can be ingested on a daily basis, as a drink by blending in green tea, black tea, oolong tea or cereal tea, or as a food by blending in biscuits, breads or candies. It can also be utilized as so-called supplement in an appropriate dosage form according to pharmaceutical dispensation described below.

When preparing as a medicament, it can be combined with appropriate pharmaceutical additives and used in various dosage forms according to conventional dispensation procedures. Such dosage forms include oral dosage forms such as, for example, solid preparation such as powder, granules, capsules, pills and tablets, liquid such as pharmaceutical solution, suspension and emulsion.

When the composition of the present invention is used as food, the use include not only a use as a common food and drink, but also use as a functional health food which exerts a certain function to contemplate health promotion.

Specific forms of this case include supplements in forms of capsules, tablets, powder, granules, etc., bakery foods such as breads, cakes and cookies, condiments such as sauce, soup, dressing and mayonnaise, dairy products such as milk, yogurt and cream, confectionery such as chocolates and candies, or various drinks such as green tea, black tea, oolong tea, barley tea, cereal tea, fruit juice, vegetable drinks, milk beverages, refreshing beverages and carbonated beverages, containing the composition of the present invention as an effective ingredient.

The dosage of the composition of the present invention when it is used as the effective ingredient of a medicament composition varies depending on the percentage of each ingredient, and also depending on various factors such as the age, body weight and gender of the patient, symptoms or the method of administration In the case of an oral administration to an adult, it can generally be selected such that the choline ester content is in a range between 5 μg and 50 mg, or in one embodiment in a range between 5 μg and 500 pg per day. The dosage can appropriately be increased or decreased depending on the level of improvement in symptoms. As for the number of administration, it can be administered once a day or divided in several times a day.

The intake of the composition of the present invention when it is used as food can be selected according to the above-described case of oral administration of a medicament. Note that the case of food and drink is different from the case of medicament in that the dose and number of administration are not limited, and therefore the intake may be selected without being limited to the above-described range in consideration of the purpose of health maintenance as well as taste and palatability, as long as it will not cause a particularly severe symptom.

### EXAMPLES

1. Test and placebo foods
(1) Preparation of the test food (choline ester-containing composition)

Harvested aubergines were heat-sterilized (90° C., 10 min), subsequently extracted to yield juice, to which an excipient (dextrin) was added at 25% to the weight of the extracted juice, freeze-dried to prepare a 25% dextrin-mixed aubergine freeze-dried powder. The functional ingredient in the 25% dextrin-mixed aubergine freeze-dried powder was quantified, and a predetermined weight of 300 mg was filled in an opaque capsule (made of hydroxypropylmethykellulose). The choline ester content per one capsule was 576 μg.
(2) Preparation of the placebo food

An opaque capsule that was the same as the test food was filled with 280 mg of excipient (dextrin).
2. Subjects

The test food and placebo food were taken by 21 male and female subjects at the ages from 30's to 50's.
3. Electroencephalogram measurement and test food ingestion
(1) Test period (schedule)

According to the schedule shown in Table 2, electroencephalogram was measured for 14 days. Ingestion of the test food and placebo food was carried out by crossover method of 10 days in total including 5 days of test food ingestion and 5 days of placebo food ingestion.

TABLE 2

| | |
|---|---|
| Day 1 and Day 2 | Electroencephalogram measurement only |
| Day 3 to Day 7 | Test or placebo food ingestion, and |
| | electroencephalogram measurement |
| Day 8 and Day 9 | Wash-out period (electroencephalogram |
| | measurement only) |
| Day 10 to Day 14 | Crossover of test or placebo food ingestion, |
| | and electroencephalogram measurement |

(2) Ingestion

6 capsule a day of the test food (choline ester amount total 3456 μg/day) or placebo food were ingested with water or warm water from 30 minutes to 1 hour before turning in. Note that the subject is principally prohibited to take alcohol drink during the test period, and prohibited to take caffeine-containing drinks after dinner to turning in. Moreover, it was determined that aubergines and processed food thereof, bamboo shoots and processed food thereof, fermented foods derived from plant materials shall not be taken.
(3) Electroencephalogram measurement

Electroencephalogram was measured by wearing a electroencephalograph (Tradename: SLEEPSCOPE (SleepWell)) during sleep. From the measurements from the first sleep cycle to the forth sleep cycle, average values were calculated for each item shown in Table 3.
4. Results

The results are shown in Table 3.

TABLE 3

| | Test food- | Placebo food- | |
|---|---|---|---|
| | ingested group | ingested group | |
| | (n = 63) | (n = 63) | t-test |
| Measurement recording time | 376.70 | 363.82 | n.s. |
| (TIB) (min) | | | |
| Sleeping latency (min) | 17.142 | 15.356 | n.s. |
| non-REM deep sleeping | 20.717 | 23.510 | n.s. |
| latency (min) | | | |
| REM sleeping latency (min) | 66.640 | 66.864 | n.s. |
| Sleeping time (min) | 356.01 | 341.88 | * |
| Total sleeping time (min) | 328.02 | 315.32 | * |
| Stage-total time (non-REM 1) | 68.568 | 62.500 | * |
| (min) | | | |
| Stage-total time (non-REM 2) | 160.73 | 159.44 | n.s. |
| (min) | | | |
| Stage-total time (non-REM 3) | 25.526 | 25.070 | n.s. |
| (min) | | | |
| Stage-total time (REM) (min) | 66.798 | 66.698 | n.s. |
| Stage-total time (awake) (min) | 26.863 | 25.734 | n.s. |

*: P < 0.05;
n.s.: non-significant

5. Consideration

As shown in Table 3, in the test food-ingested group, sleeping time was significantly increased as compared to the placebo food-ingested group. In particular, non-REM total time, which is an index of deep sleep was significantly increased. Because there was no significant difference in sleep efficiency, it became clear that the sleeping time was increased not due to a deterioration in the sleep efficiency, but sleep time could be increased while keeping the sleep efficiency. Moreover, increased percentage of deep sleep clarified an effect of improving sleep rhythm. By these effects, an improvement in sleep was recognized.

Moreover, an improved sleep efficiency, shortened sleep induction time, increased non-REM sleeping time or reduced nocturnal awakening can be expected by a method of food ingestion with reduced burden such as, for example, decreased number of capsules to be taken at once,

### INDUSTRIAL APPLICABILITY

The composition of the present invention has a remarkable sleep-improving effect, and by including this as an active ingredient, a food with functional claims or a medicament for treating sleep disorder, etc. can be produced.

1. A method for improving sleep comprising orally administrating a composition comprising a choline ester as an effective ingredient to a subject.

2. The method according to claim 1, wherein the composition is a food composition.

3. The method according to claim 1, wherein the choline ester is derived from a food plant.

4. The method according to claim 3, wherein the food plant is a fruit of the family Solanaceae, the genus Solanum, the species Solanum melongena and/or a young bud of the family Poaceae, the subfamily Bambusoideae, the tribe Bambuseae.

5. The method according to claim 1, wherein the choline ester comprises one or more selected from the group consisting of acetylcholine, butylcholine and propionylcholine.

6. The method according to claim 5, wherein the choline ester does not comprise lactoylcholine.

7. The method according to claim 1, wherein daily intake of the choline ester is between 7.5 μg and 750 mg.

8. The method according to claim 1, wherein the improving sleep is an increase in sleeping time.
